(19) United States
(12) Patent Application Publication
(10) Pub. No.: US 2023/0058634 A1
(43) Pub. Date: Feb. 23, 2023

(54) PREVENTION OF PLATELET ACTIVATION AND CELL DIFFERENTIATION DURING THE PROCESSING OF BLOOD AND BLOOD-RELATED COMPOSITIONS

(71) Applicant: GPB Scientific, Inc., Richmond, VA (US)

(72) Inventors: Roberto CAMPOS-GONZALEZ, Carlsbad, CA (US); Khushroo GANDHI, Palo Alto, CA (US); Alison SKELLEY, Riverside, CA (US); Anthony WARD, Rancho Santa Fe, CA (US); Yasna BEHMARDI, Mission Viejo, CA (US)

(21) Appl. No.: 17/793,626

(22) PCT Filed: Jan. 19, 2021
(86) PCT No.: PCT/US2021/013902

§ 371 Date: Jul. 18, 2022

Related U.S. Application Data
(60) Provisional application No. 62/963,362, filed on Jan. 20, 2020.

(51) Int. Cl. C12N 5/0783; A61P 37/02

(52) U.S. Cl. CPC C12N 5/0636; A61P 37/02; C12N 2510/00

(57) ABSTRACT

The present invention is directed to improved methods of preparing cells and compositions for therapeutic uses. In particular, the invention is concerned with methods which inhibit platelet activation and/or cell differentiation during the processing of blood and blood-related compositions.

### CROSS REFERENCE TO RELATED APPLICATIONS

The present application claims the benefit of U.S. Provisional Patent Application No. 62/963,362, filed on Jan. 20, 2020.

### FIELD OF THE INVENTION

The present invention concerns the use of blood and blood-related compositions in the preparation of cells for therapeutic use and especially methods and compositions that can be used to inhibit platelet activation and/or cell differentiation during this process.

### BACKGROUND OF THE INVENTION

Contrary to venous blood collection, apheresis perturbs blood components resulting in many changes, including the activation of platelets. Factors released by these platelets, as well as other factors in the blood may cause cells of therapeutic interest to divide and differentiate. For example, naive T cells may be induced to develop into T memory stem cells, followed by central memory T cells, effector memory cells and finally short lived effector T cells (see Gattinoni, et al.; Moving T memory stem cells to the clinic, Blood 121(4):567-568 (2013)). Since early stage cells are often more desirable for therapeutic applications, methods for eliminating or inhibiting platelet activation and for limiting the effect of factors promoting cell division and development are of considerable interest.

### SUMMARY OF THE INVENTION

In a first aspect, the invention is directed to a method for producing a population of therapeutically useful target cells from blood or a blood-related composition by: a) obtaining the blood or blood-related sample composition comprising the target cells; b) during or after the obtaining of the blood or blood-related sample, adjusting the sample so that it comprises one or more exogenous agents that inhibit platelet activation and/or inhibit the action of one or more factors that contribute to the proliferation or activation of the target cells; and c) separating the target cells from platelets to obtain an enriched population of target cells. The adjustment of step b) may involve adding one or more exogeneous agents not previously in the sample collected (or being collected) or it may involve increasing the level of a component already present.

The term “blood-related composition” refers to a composition derived from blood. For example, an apheresis or leukapheresis sample would be a blood-related composition. The term “target cells” includes any cells in a sample that may be effectively used in the treatment of a patient. The target cells may be: leukocytes, including neutrophils, basophils, eosinophils, lymphocytes (including B cells, T cells and natural killer cells); monocytes; macrophages; mast cells; or dendritic cells. However, for the purposes herein, this term does not include platelets or red blood cells. The most preferred target cells are T cells. The term “exogenous agents” refers to agents that, if not adjusted, would not be in the sample, or would not be at the concentration present in the sample. For example, a synthetically produced zinc chelator might be an exogenous agent. Although citrate is present in blood, it may still be an exogenous agent if it has been added so that its concentration is higher than it would otherwise be. Numerous agents that inhibit platelet activation and/or inhibit the action of one or more factors that contribute to the proliferation or activation of the target cells are described herein and may be used as exogenous agents.

Any method that is effective at separating target cells from platelets may be used to obtain an enriched population of target cells from blood or a blood-related composition. However, deterministic lateral displacement (DLD) is the most preferred method and centrifugation methods should generally be kept to a minimum or avoided. The DLD device should comprise an array of obstacles arranged in rows, with each subsequent row of obstacles shifted laterally with respect to a previous row. The obstacles are positioned so as to differentially deflect target cells to a first outlet where they may be recovered as a product, and to direct platelets to a second outlet where they may be collected or discarded as waste. Other microfluidic or non-microfluidic procedures that separate on the basis of size may also be used, as well as affinity methods, methods that separate using magnetic, inertial or acoustic fields, methods using hydrophobic interactions, approaches using microbubbles or buoyant beads etc. The separation should reduce the platelets by at least 70% (and preferably by 80%, 90% or 95%) compared to the sample before separation and/or reduce the ratio of platelets to total target cells by at least 70% (and preferably by 80%, 90% or 95%).

The exogenous agents used in the method should be at a concentration such that an enriched population of target cells is produced in which there is an increase in earlier stage cells and/or cells that have undergone fewer divisions compared to a sample prepared in the same way but in the absence of the exogenous agent or agents. For example, there may be more naïve T cells than effector T cells or memory T cells. The ratio of non-terminally differentiated target cells (NTD) to terminally differentiated target cells (TD) or target cells at a later stage of differentiation (LSD), NTD/TD or NTD/LSD, should be at least 20% (and preferably at least 50%, 100% or 500%) greater than in a sample prepared in the same way but in the absence of the exogenous agents. In some cases, compared to cells prepared in the absence of one or more exogenous agents, the number of naïve T cells may be, for example, at least 2, 3, 5, 10, 20 or 100 times higher than the total number of cells present or the total number of T cells present in the enriched population of cells.

Once an enriched population of target cells is obtained, the cells may be genetically engineered to have a therapeutically useful phenotype. Any method of engineering the cells may be used, including transfection and viral transformation. By way of example, enriched T cells (preferably naive T cells, T memory stem cells, or central memory T cells) may be engineered to express chimeric antigen receptors, expanded and used therapeutically as CAR T cells in the treatment of a patient for cancer, an autoimmune disease or an infectious disease.

The use of exogenous agents that minimize target cell differentiation and/or proliferation may be used in conjunction with other methods designed to limit cell division. For example, in procedures involving the genetic engineering of target cells, cell division will typically be required for recombinant sequences to be incorporated into a cell's genome. However, the number of divisions may be minimized, and preferably limited to an average of just two (more preferably just one), by initially avoiding cell division as described herein and then adding an agent that promotes cell division shortly prior to genetic engineering. Factors promoting cell division include agents released by immune cells, cytokines, peptides, peptide-receptor complexes, and antibodies either alone or in conjunction with other costimulatory molecules. Such agents should not be added more than two or three days before transformation is initiated and their addition may be delayed to one day (or alternatively, 5 hours, three hours or one hour) before recombinant transformation is initiated. Once genetic engineering has been completed, the agent or agents promoting cell division should be blocked or separated from the engineered target cells, preferably by DLD. Although freezing should preferably be avoided, target cells can be preserved in this manner prior to or after genetic engineering.

The total number of divisions that a non-cancerous cell can undergo is limited. For example a typical T cell has the capacity to double approximately 20 times during its lifespan. However, once it has terminally differentiated it is capable of only 3-5 more doublings (PNAS 92:3707-3711 1995; Immunity 44:380-390 2016). One of the main advantages of minimizing the number of cell doublings during processing is that it allows the production of therapeutic cells that can divide more times after being administered to a patient. Using the methods described herein target cells should preferably undergo no more than 3 (and more preferably no more than 2 and or 1) doublings from the time that the blood or blood-related sample composition is obtained until genetic engineering of cells is complete.

After enrichment and/or after genetic transformation, target cells may be expanded in culture to obtain sufficient cells for the treatment of a patient. Expansion would typically occur, for example, in cases where the target cells are T cells that have been genetically transformed to express chimeric antigen receptors, i.e., in cases where CAR T cells are being made. Using the methods described herein, after expansion in culture is complete, target cells should have undergone no more than 6 (and preferably no more than 5, 4 or 3) doublings.

More generally, the therapeutic products made should preferably comprise a population of target cells, e.g., T cells, that have undergone no more than 5 (and more preferably no more than 4 or 3) doublings. As a result, after administration to a patient, the cells should preferably be capable of at least 10 (and more preferably 15 or 17) doublings before reaching senescence. Most preferably, the target cells in the therapeutic product will be CAR T cells. Thus, the invention encompasses therapeutic compositions comprising CAR T cells that have undergone no more than 5 (and preferably no more than 4 or 3) doublings and/or that, after administration to a patient, are capable of at least 10 (and preferably 15 or 17) doublings before reaching senescence.

In some embodiments, the blood or blood-related sample composition has a white blood cell content of between 0.5×10⁹ and 13×10⁹ cells. Preferably at least 75% of these cells are recovered in the enriched population of target cells with a higher recovery (80% or 90%) being preferred.

The exogenous agents should preferably be maintained during processing at least until the time that the cells are genetically engineered and should preferably be added during the time the sample composition is collected or before collection if possible.

In the methods described above, the one or more exogenous agents may be selected from the group consisting of: a gpIIb/IIIa inhibitor (e.g., tirofiban); GSK484; the non-permeable zinc-chelator DTPA; alexidine; Y-27632; MRS2578; rivaroxaban; Dextran-40; and combinations thereof. When present, a gpIIb/IIIa inhibitor; GSK484; DTPA; alexidine; Y-27632; MRS2578; or rivaroxaban should typically be present in the sample composition of step b), and/or the enriched population of target cells of step c), at a concentration of from 0.5 μg/ml to 100 μg/ml. When Dextran-40 is present it should be at a concentration of from 0.1% to 10% by weight and when the exogenous agent is sodium citrate, its final concentration in the sample composition of step b), and/or the enriched population of target cells of step c) should be from 10 mM to 40 mM.

### BRIEF DESCRIPTION OF THE DRAWINGS

FIGS. 1-10 are described below. These figures and the accompanying descriptions are provided solely for illustrative purposes.

FIG. 1: shows red blood cell to white blood cell ratios and platelet to white blood cell ratios across multiple apheresis products and different cellularities (X axis). Leukopacks are depicted on the left and residual leucocyte fractions (LRS) are on the right.

FIG. 2: depicts a multiplex analysis of cytokines and growth factors from apheresis input, after DLD and Ficoll. An apheresis sample is centrifuged to remove all cellular and particulate components (source). The fractions are debulked by DLD and Ficoll and are centrifuged to remove cells and particulates. All three supernatants derived from source, DLD and Ficoll separations, are analyzed by a multiplex assay to determine the concentrations of cytokines and growth factors. The figure suggests that the DLD fraction has a lesser concentration of the analytes tested. Remarkably, the Ficoll fraction shows a significant amount of several cytokines like IL-8, sCD40L, and Fractalkine in addition to PDGF and TGFbeta, all of them inducers of an immunosuppressive environment.

FIG. 3: illustrates ways in which an apheresis collection procedure may release multiple proinflammatory factors. The process may lead to a loss of naive CD4 cells available for the production of CAR-T cells.

FIG. 4: shows a determination of T cell subtypes before (input) and after (debulked) separation by DLD and Ficoll using flow cytometry gating on CD3+ cells (T) in combination with CD197 and CD45RA staining. CD45RA−/CD197− (T effector memory); CD45RA+/CD197− (T effector); CD45RA−/CD197+ (T central memory); CD45RA+/CD197+ (T naïve). Both separation processes result in similar proportions of T cell subtypes.

FIG. 5: An apheresis product is collected into two different Control formulations, one with 14 mM Citrate (ACD-A) and one with 28 mM Citrate (2x ACD-A). Aliquots of each are incubated overnight with different concentrations of inhibitors (low, 1 micromolar; medium, 5 micromolar; and high, 50 micromolar; except for GSK484 in which low is 0.1 micromolar; medium is 1 micromolar; and high is 10 micromolar). Controls do not have any compound. In FIG. 5, apheresis material collected in 28 mM citrate shows less cell aggregation compared with collection at 14 mM citrate and this is independent of the inhibitor concentration. In the 28 mM Citrate sample, the combination of GSK484 and DTPA and the mixture of Tirofiban+ GSK484 improves the % of singlets (less aggregation). In the 14 mM Citrate all inhibitors, and combinations, improve the % of singlets above the control. Determination of singlets and aggregates may be done by flow cytometry using side and forward scatter of the CD45+ cell population.

FIG. 6: An apheresis product is collected into two different Control formulations, one with 14 mM Citrate (ACD-A) and one with 28 mM Citrate (2x ACD-A). Aliquots of each are incubated overnight with different concentrations of inhibitors (low, 1 micromolar; medium, 5 micromolar; and high, 50 micromolar; except for GSK484 in which low is 0.1 micromolar; medium is 1 micromolar; and high is 10 micromolar). In FIG. 6, viability is higher in apheresis collected in 28 mM citrate than 14 mM citrate. The presence of inhibitors in the 28 mM citrate sample does not affect viability. Cell viability may be determined by flow cytometry using CD45-PerCP and 7-AAD.

FIG. 7: An apheresis product is collected into 14 mM Citrate (ACD-A). Aliquots are incubated overnight with different concentrations of inhibitors (low, 1 micromolar; medium, 5 micromolar; and high, 50 micromolar; except for GSK484 in which low is 0.1 micromolar; medium is 1 micromolar; and high is 10 micromolar). The calcium ionophore A23187 is used at 4 micromolar in apheresis material resuspended in DMEM media. Singlet analysis (top panel) shows that netosis inhibitors decrease aggregation (increased singlets %) compared to the control with no inhibitors. GSK484 and MRS2578 inhibit aggregation in a dose dependent manner whereas DTPA is very effective at all concentrations tested. None of the inhibitors affect viability (lower panel) except for the calcium ionophore A23187 control. Determination of singlets and aggregates may be done by flow cytometry using side and forward scatter of the CD45+ cell population. Cell viability is determined as the % CD45+ cells.

FIG. 8: An apheresis product is collected, split and collected into two different Control solutions, one with 14 mM Citrate (1X ACD-A) and one with 28 mM Citrate (2X ACD-A) plus Tirofiban 2.5 micrograms/ml and DTPA at 10 micromolar final concentration. The containers are maintained under rotation at room temperature overnight. At 24 h, aliquots are taken from each fraction and stained for the presence of white blood cells (CD45) and platelets (CD41) and counterstained with DAPI for nuclear staining. FIG. 8 shows that the collection in extra ACD and in the presence of Tirofiban and DTPA diminishes the aggregation of platelets.

FIG. 9: An apheresis product is collected in 14 mM Citrate and divided into two different tubes—one with no further addition and a second tube with additional citrate for a final concentration of 28 mM (2X ACD-A). The tubes are left overnight at room temperature under rotation. After 24 h, aliquots of the tubes are spotted on a glass slide and are analyzed under the microscope for the presence of cell aggregates. FIG. 9 shows that the addition of extra citrate prevents the formation of cell aggregates.

FIG. 10: An apheresis product is collected in 14 mM Citrate (ACD-A) and aliquots are taken and incubated with the different inhibitors, or in combination, in different tubes for 24 h. The tubes are left under rotation at room temperature. After 24 h, aliquots of the different tubes are spotted on a glass slide and are analyzed under a microscope for the presence of cell aggregates. The different inhibitors are GSK484 at 10 micromolar, DTPA 10 micromolar, Tirofiban 2.5 micrograms/ml. FIG. 10 shows that the addition of Tirofiban or Tirofiban in combination with DTPA and/or GSK484 prevents the formation of cell aggregates. The combination of FTPA and GSK484 also prevents the formation of cell aggregates.

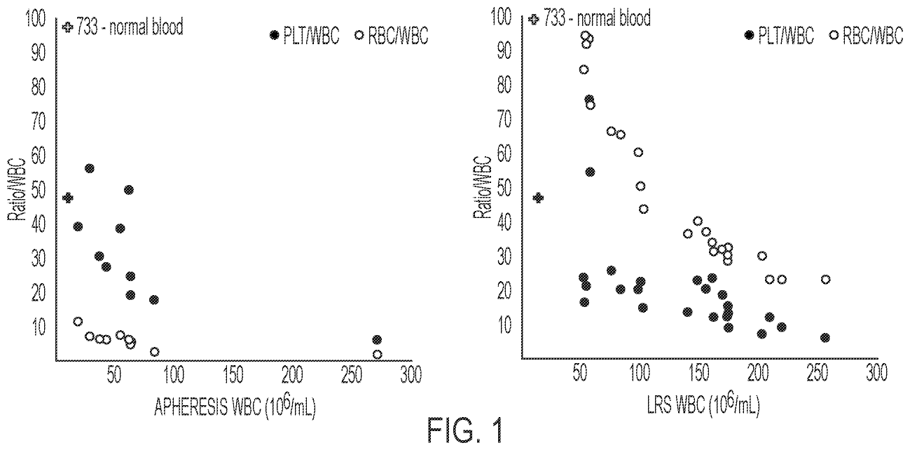

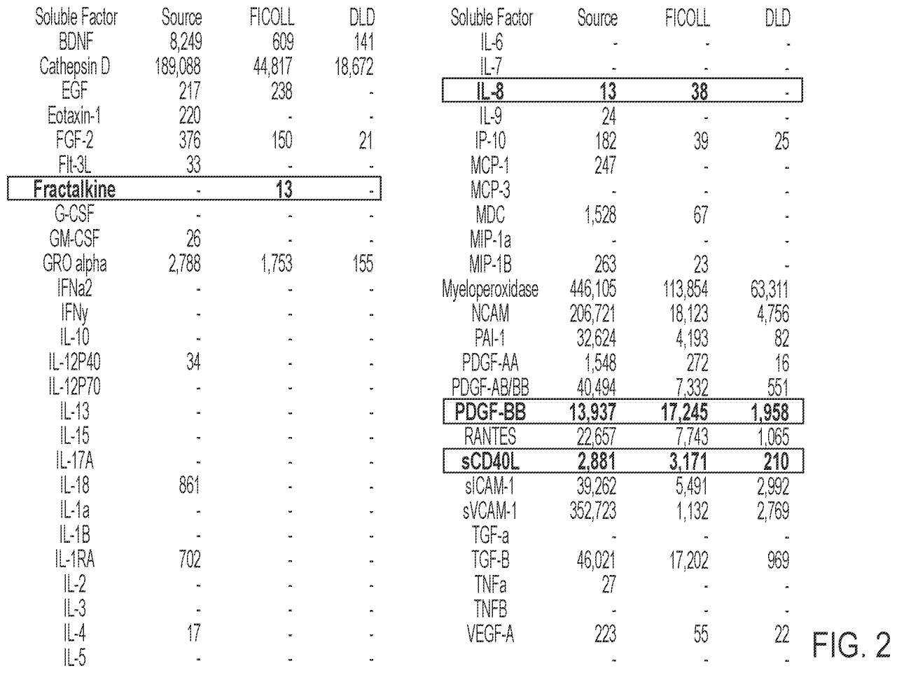

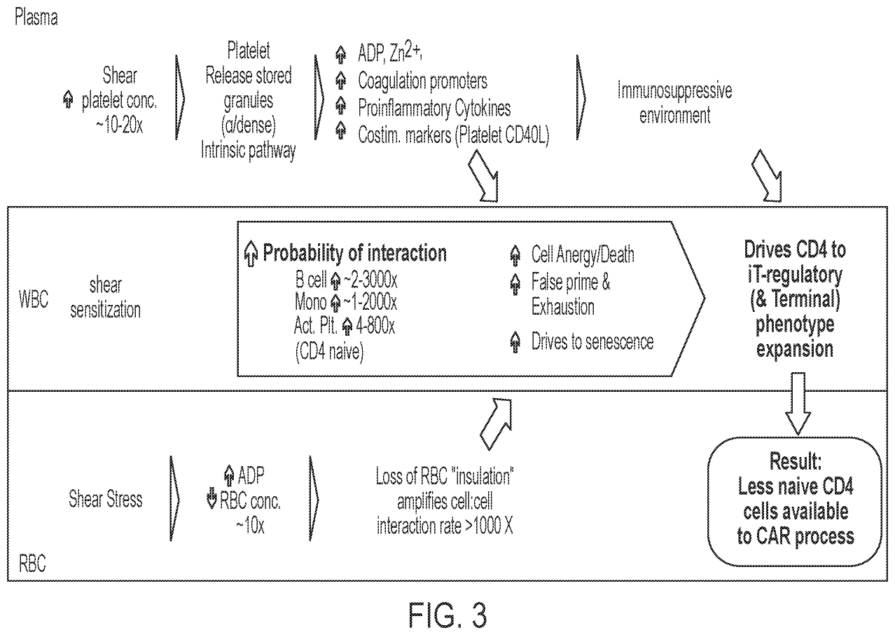

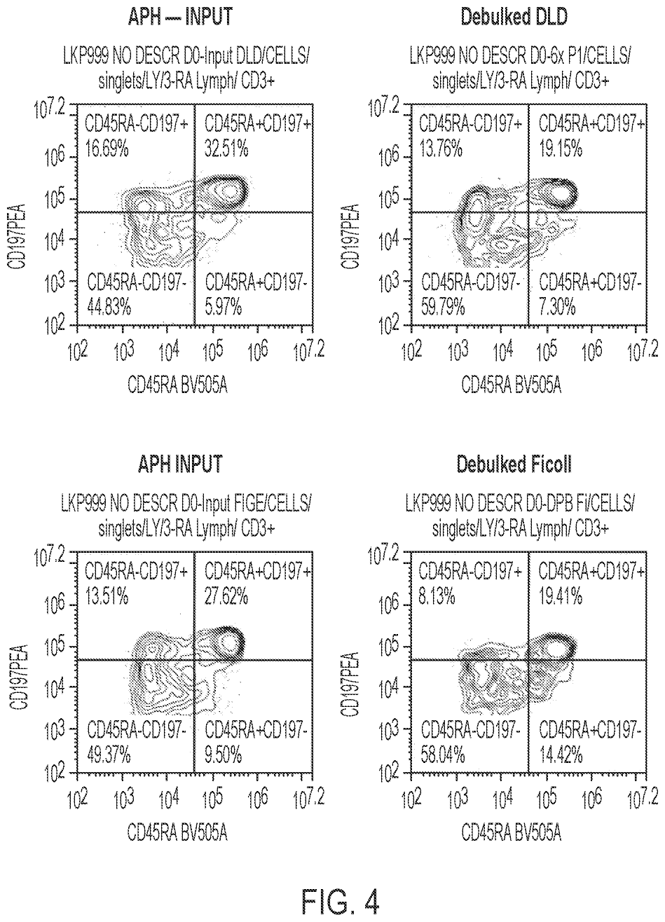

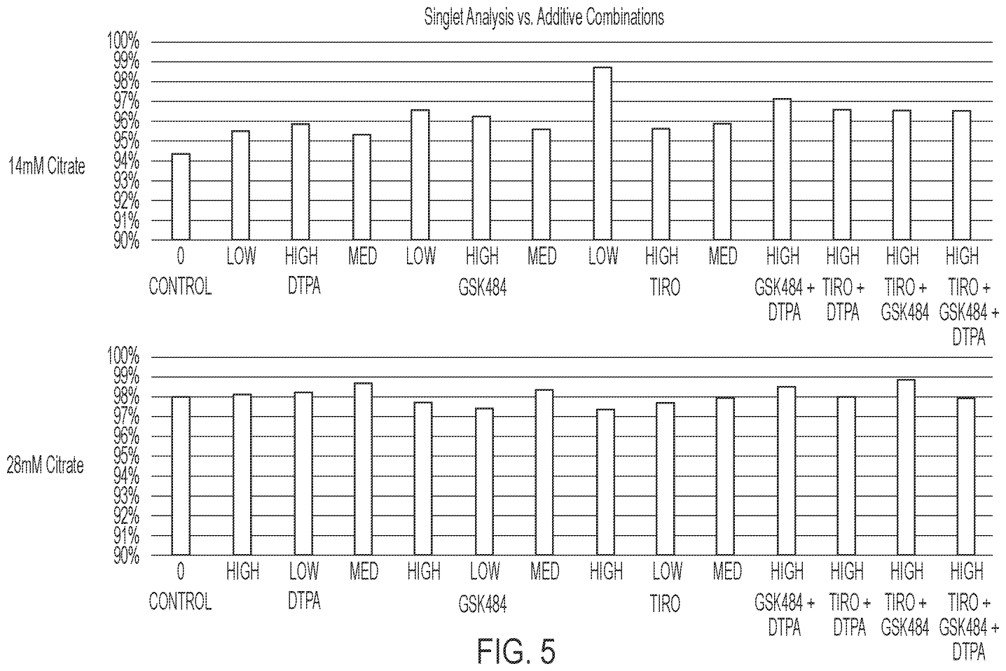

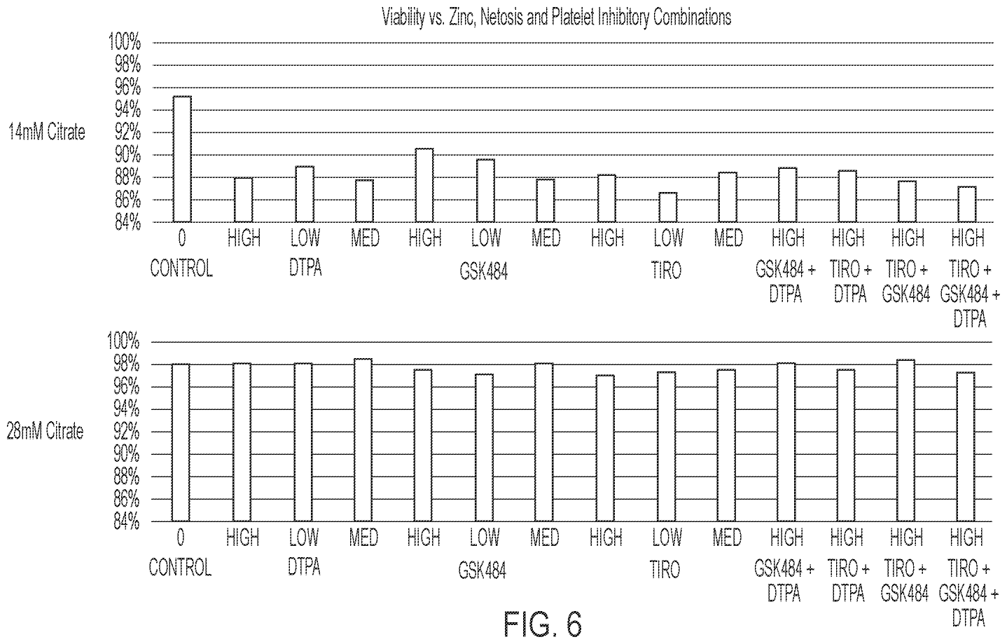

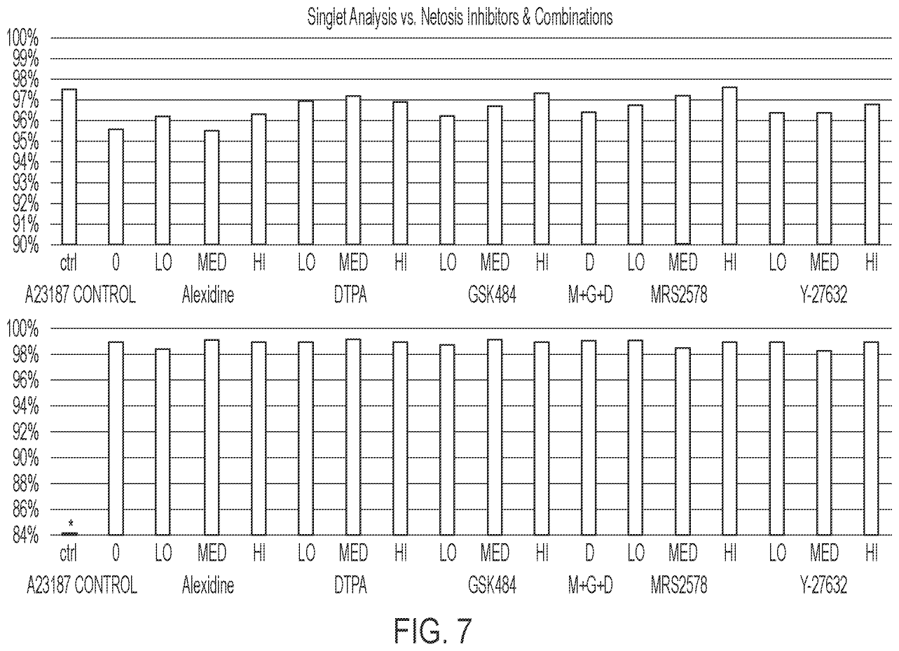

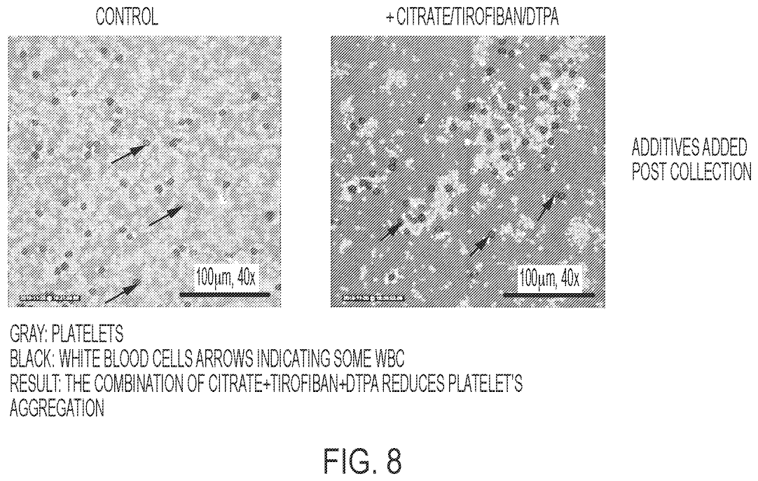

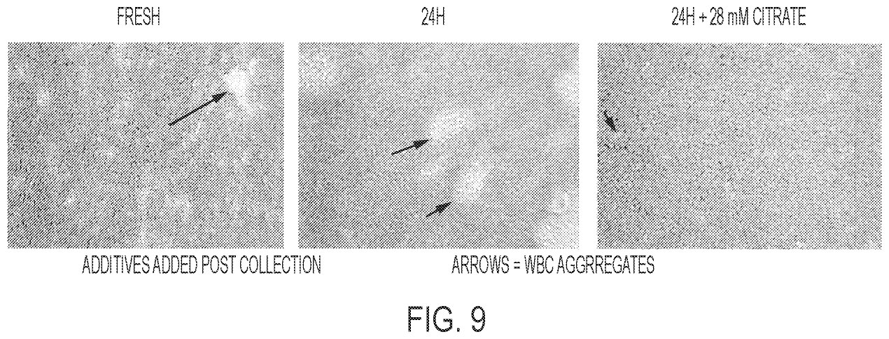

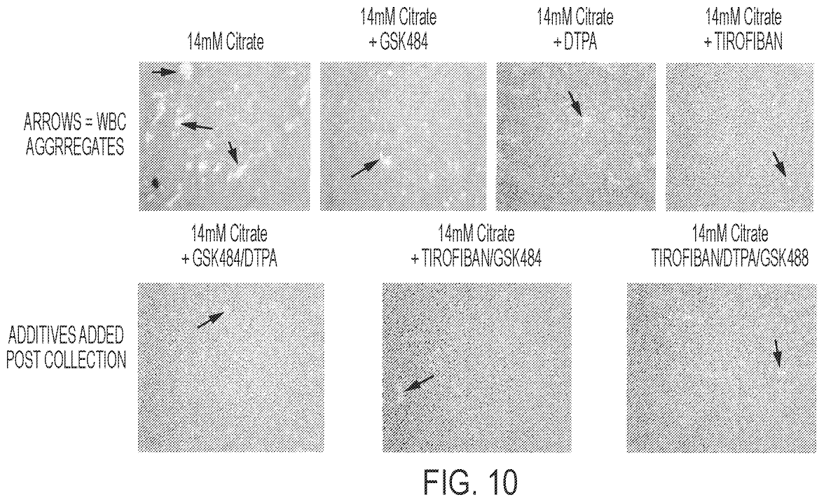

### DEFINITIONS

Apheresis: As used herein this term refers to a procedure in which blood from a patient or donor is separated into its components, e.g., plasma, white blood cells and red blood cells. More specific terms are “plateletpheresis” (referring to the separation of platelets) and “leukapheresis” (referring to the separation of leukocytes). In this context, the term “separation” refers to the obtaining of a product that is enriched in a particular component compared to whole blood and does not mean that absolute purity has been attained.

CART cells: The term “CAR” is an acronym for “chimeric antigen receptor.” A “CAR T cell” is therefore a T cell that has been genetically engineered to express a chimeric receptor.

CART cell therapy: This term refers to any procedure in which a disease is treated with CAR T cells. Diseases that may be treated include hematological and solid tumor cancers, autoimmune diseases and infectious diseases.

Carrier: As used herein, the term “carrier” refers an agent, e.g., a bead, or particle, made of either biological or synthetic material that is added to a preparation for the purpose of binding directly or indirectly to cells present. Carriers may be made from a variety of different materials, including DEAE-dextran, glass, polystyrene plastic, acrylamide, collagen, and alginate and will typically have a size of 1-1000 μm. They may be coated or uncoated and have surfaces that are modified to include affinity agents that recognize antigens or other molecules on the surface of cells. The carriers may also be magnetized and this may provide an additional means of purification to complement DLD.

Carriers that bind “in a way that promotes DLD separation”: This term, refers to carriers and methods of binding carriers that affect the way that a cell behaves during DLD. Specifically, “binding in a way that promotes DLD separation” means that: a) the binding must exhibit specificity for a particular target cell type; and b) must result in a complex that provides for an increase in size of the complex relative to the unbound cell. In cases where therapeutic or other uses require that target cells be released from complexes to fulfill their intended use, then the term “in a way that promotes DLD separation” also requires that the complexes permit such release.

Target cells: As used herein “target cells” are the cells that various procedures described herein require or are designed to purify, collect, engineer etc. What the specific cells are will depend on the context in which the term is used.

Isolate, purify: Unless otherwise indicated, these terms, as used herein, are synonymous and refer to the enrichment of a desired product relative to unwanted material. The terms do not necessarily mean that the product is completely isolated or completely pure. For example, if a starting sample had a target cell that constituted 2% of the cells in the sample, and a procedure was performed that resulted in a composition in which the target cell was 60% of the cells present, the procedure would have succeeded in isolating or purifying the target cell.

Bump Array: The terms “bump array” and “obstacle array” are used synonymously herein and describe an ordered array of obstacles that are disposed in a flow channel through which a cell or particle-bearing fluid can be passed.

Deterministic Lateral Displacement: As used herein, the term “Deterministic Lateral Displacement” or “DLD” refers to a process in which particles are deflected on a path through an array, deterministically, based on their size in relation to array parameters. This process can be used to separate cells, which is generally the context in which it is discussed herein. However, it is important to recognize that DLD can also be used to concentrate cells and for buffer exchange.

Critical size: The “critical size” or “predetermined size” of cells passing through an obstacle array describes the size limit of particles that are able to follow the laminar flow of fluid. Particles larger than the critical size can be ‘bumped’ from the flow path of the fluid while particles having sizes lower than the critical size (or predetermined size) will not necessarily be so displaced. When a profile of fluid flow through a gap is symmetrical about the plane that bisects the gap in the direction of bulk fluid flow, the critical size can be identical for both sides of the gap; however when the profile is asymmetrical, the critical sizes of the two sides of the gap can differ.

Fluid flow: The terms “fluid flow” and “bulk fluid flow” as used herein in connection with DLD refer to the macroscopic movement of fluid in a general direction across an obstacle array. These terms do not take into account the temporary displacements of fluid streams for fluid to move around an obstacle in order for the fluid to continue to move in the general direction.

Tilt angle ε: In a bump array device, the tilt angle is the angle between the direction of bulk fluid flow and the direction defined by alignment of rows of sequential (in the direction of bulk fluid flow) obstacles in the array.

Array Direction: In a bump array device, the “array direction” is a direction defined by the alignment of rows of sequential obstacles in the array. A particle is “bumped” in a bump array if, upon passing through a gap and encountering a downstream obstacle, the particle's overall trajectory follows the array direction of the bump array (i.e., travels at the tilt angle relative to bulk fluid flow). A particle is not bumped if its overall trajectory follows the direction of bulk fluid flow under those circumstances.

### DETAILED DESCRIPTION OF THE INVENTION

### I. Factors Contributing to Platelet Activation and Aggregation

Under normal physiological plasma calcium concentrations, injury to blood vessels initiates a calcium-dependent cascade of events leading to platelet activation and aggregation, activation of thrombin and ultimately the formation of a plug due to the conversion of fibrinogen into fibrin. Independently of calcium, when a lesion occurs, tissue proteins and factors are released into the blood vessel. In addition to the presence of these factors, the serum protein von Willebrand Factor (vWF) responds to a change in local shear rates by physically unrolling, exposing a binding region specific for both damaged tissue as well as platelets, and creating a vWF:Platelet bridge at the site of the lesion via a Gplb receptor. This reversible binding event causes platelet activation, and also the release of thrombin.

Shear stress and hydrodynamic forces also exert effects on platelets inducing activation-independent aggregation by the binding of the platelet surface protein GIbα to the A1 domain of the von Willebrand protein, resulting in the thrombus formation. Additional platelet surface proteins like the integrin α_{II}β₃ also bind plasma proteins like fibrinogen, von Willebrand factor (VWF), collagen and fibronectin among others. This platelet-plasma protein interaction recruits more platelets thus inducing aggregation and eventually forming a plug at the site of the injury.

Upon binding and surface activation, platelets release their intracellular contents, or granules, containing pro-thrombin, zinc, thromboxane A2, ADP, etc., thereby amplifying the calcium-dependent coagulation cascade and other pathways ultimately leading to platelet aggregation. By promoting the binding of fibrinogen to the platelet surface protein α_{II}β₃, zinc promotes platelet aggregation. Zinc binds numerous plasma binding proteins, including Factor XII/XIIa, Factor VIIa, Fibrinogen, etc., affecting blood hemostasis. For example, the binding of zinc to Factor XII/XIIa enhances its autoactivation resulting in coagulation. Both EDTA and ACD are poor zinc chelators at routine blood and apheresis collections necessitating the addition of specific zinc removal mechanisms to minimize zinc effects.

### II. Factors of Interest in Therapeutic Cell Recovery

NETosis Inhibitors: NET or neutrophil extracellular traps, or DNA expulsion, are extracellular DNA extensions with granular proteins which are formed during the contact of neutrophils with bacteria, activated platelets, or fungi. This contact results in the death of the neutrophil and the trapping and killing of the invaders. Death by NET formation is different from apoptosis and necrosis. NETs also participate in clot formation resulting from damage to blood vessels, contact activation of platelets leading to the release of proteases and chromatin from neutrophils. NETosis relies on the adhesion of neutrophils through their integrin surface receptors like Mac-1. Further, NETosis can be induced by hemodynamic shear forces and has been implicated in diseases such as inflammation and autoimmune diseases. Although NET formation was first described in neutrophils, this form of cell death has also been observed in mast cells and eosinophils. NET formation in monocytes is initiated by several different pathways, like calcium and ion surges inside the cell, increases of pH, ROS (reactive oxygen-species) formation. NET's are induced by lipopolysaccharides, IL-8 and GMCSF. NET's have also been demonstrated to promote platelet aggregation. NETosis is a caspase-independent process and regulated by PAD4, a protein arginine deiminase. PAD catalyzes the citrullination of arginines in chromatin proteins. Several inhibitors of NETosis are described below.

gpllb/IIIa inhibitors: This is a class of inhibitors exemplified by tirofiban, a potent reversible inhibitor of the platelet surface protein α_{II}β₃. α_{II}β₃ is a heterodimeric surface protein that binds, in an activation dependent-manner to plasma proteins like von Willebrand Factor, fibrinogen and prothrombin. This small compound blocks the interaction between the platelet's α_{II}β₃ and its ligand proteins preventing the aggregation of platelets. Tirofiban's structure mimics the RGD-motif found in the α_{II}β₃-ligands. There are other inhibitors of the surface protein α_{II}β₃ that impede platelet aggregation such as the monoclonal antibody Abciximab, and chemical compounds like Eptifibatide, Roxifiban and Orbofiban, among others.

Dextran-40: Dextran-40 reduces the heparin-mediated aggregation of platelets and reduces spontaneous and agonist-induced platelet aggregation as well as the surface expression of markers of platelet activation. Furthermore, Dextran-40 inhibits the binding of platelets to von Willebrand protein in vivo, by inducing the breakdown of vWF and prevents platelet activation by cleaving the thrombin receptor.

Rivaroxaban: Rivaroxaban is an inhibitor of Factor Xa, in both intrinsic and extrinsic pathways, of the coagulation cascade. Rivaroxaban blocks the formation of thrombin by binding and inhibiting the pro-thrombin protein complex. Further, by blocking Factor Xa, it prevents platelet activation by PAR-1 (protease activated receptor-1).

Zinc: It has been demonstrated that zinc is a potent inducer of platelet aggregation at the sub-millimolar concentrations found in plasma. Zinc also potentiates ADP-induced platelet aggregation. Further, Zinc enhances the aggregation induced by the activation of the α_{II}β₃ pathway by promoting the recruitment of fibrinogen. It appears zinc's effects are mediated by at least two different mechanisms. One is by an increase of the intracellular Zinc concentration in platelets leading intracellular tyrosine phosphorylation of multiple platelet proteins. The second mechanism, is by zinc's changing the conformation and activation of key proteins in the coagulation cascade.

Zinc chelators like DTPA (diethylenetriaminepentacetic acid) and TPEN (N,N,N′, N′-tetrakis(2-pyridylmethyl)ethylenediamine) remove Zinc from biological samples like plasma. Whereas TPEN is a membrane-permeable zinc chelator, DTPA does not cross the plasma membrane. At micromolar concentrations, both chelators induce apoptosis and cell death in cultured cells and the DTPA-cytotoxic effect can be blocked by N-acetyl-L-cysteine indicating a redox mechanism of action for this inhibitor.

GSK484: GSK484 is an inhibitor of the human and mouse PAD4, a protein arginine deiminase that is predominantly expressed in granulocytes. PAD4 and other PAD's are involved in histone citrullination and NET (neutrophil extracellular trap) formation. GSK484 has minimal activity towards other enzymes. It inhibits PAD4 with an IC50 of 50 nM in the absence of calcium. GSK484 will prevent NET formation during apheresis due to neutrophil activation and death.

Y-27632: Y-27632 is a ROCK kinase inhibitor that improves the recovery of cryopreserved cells after freezing and thawing. ROCK kinase is a key enzyme that regulates cell shape and movement through its effects on cytoskeletal proteins. In addition, pre-treatment with Y-27632 inhibits NET formation induced by PMA and other challenges. Incubation with Y27632 blocked the chemotaxis of granulocytes.

MRS-2578: MRS-2578 is a purinergic receptor (P2Y6) antagonist that blocks the activation of neutrophils thereby preventing aggregation and the formation of NET triggered by MSU crystals.

pH: Extracellular alkaline pH, i.e., above 7.2, has been demonstrated to increase calcium influx thereby inducing a concomitant increase in intracellular pH which leads to an increase in the intracellular calcium concentration and PAD activation. This results in NET formation in neutrophils.

Alexidine: Alexidine is a bisguanine compound that inhibits NET formation induced by ionomycin, in neutrophils. Interestingly, alexidine also inhibits the basal level of netosis even without an increase of cytosolic calcium. However, after 2 h of incubation alexidine induces NETosis.

TABLE 1
Summary of Factors of Interest in Cell Processing

| FACTOR | EFFECT | REF |
|---|---|---|
| GSK484 | Inhibits PAD4 preventing NETosis | 8 |
| DEXTRAN-40 | Prevents PLT aggregation and | 9, 10 |
| | activation | |
| TIROFIBAN | Inhibits α_{II}β₃ preventing PLT | 11 |
| | aggregation | |
| Sodium Citrate | Calcium chelator | Sigma- |
| Tribasic Dihydrate | | Aldrich |
| DTPA & TPEN | Zinc chelators | 12, 13 |
| Y-27632 | ROCK kinase inhibitor and prevents | 14, 15 |
| | NET formation | |
| MRS-2578 | Purinergic receptor antagonist | 16 |
| | preventing neutrophil activation and | |
| | NETosis | |
| pH 6.8 | Rising pH above 7.0 induces an | 17 |
| | increase in intracellular calcium | |
| | concentration and NET formation | |
| ALEXIDINE | Bisguanine compound that inhibits | 18 |
| | NET formation in neutrophils | |
| RIVAROXABAN | Factor Xa inhibitor preventing | 19 |
| | formation of thrombin | |

### III. Separation Methods

Although many separation methods are compatible with the invention, an especially preferred separation method is DLD. In this type of separation, microfluidic devices are characterized by the presence of at least one channel which extends from a sample inlet to one or more fluid outlets, and which is bounded by a first wall and a second wall opposite from the first wall. An array of obstacles is arranged in rows in the channel, with each subsequent row of obstacles being shifted laterally with respect to a previous row. The obstacles are disposed in a manner such that, when a crude fluid composition is applied to an inlet of the device and passed through the channel, target cells flow to one or more collection outlets where an enriched product is collected, and contaminant cells or particles flow to one more waste outlets that are separate from the collection outlets.

Once the target cells have been purified using the device, they may be transfected or transduced with nucleic acids designed to impart upon the cells a desired phenotype, e.g., to express a chimeric molecule that makes the cells of therapeutic value. The population of cells may then be expanded by culturing in vitro.

Compositions containing target cells will preferably be processed without freezing (at least up until the time that they are genetically engineered). Blood and related sample compositions will preferably be the blood (or derived from the blood) of a patient, and most preferably will be a composition containing leukocytes obtained as the result of performing apheresis or leukapheresis.

Although it is not essential that target cells be bound to a carrier before being genetically engineered, either before or after DLD is first performed (preferably before) they may be bound to one or more carriers provided that the carriers do not activate the cells. The exact means by which this occurs is not critical to the invention but binding should preferably be done “in a way that promotes DLD separation.” This term, as used in the present context, means that the method must ultimately result in binding that exhibits specificity for a particular target cell type, that provides for an increase in size of the complex relative to the unbound cell of at least 2 μm (and alternatively at least 20, 50, 100, 200, 500 or 1000% when expressed as a percentage) and, in cases where therapeutic or other uses require free target cells, that allow the target cell to be released from complexes by chemical or enzymatic cleavage, chemical dissolution, digestion, due to competition with other binders, by physical shearing, e.g., using a pipette to create shear stress, or by other means.

### IV. Designing Microfluidic Plates

Cells, particularly cells in compositions prepared by apheresis or leukapheresis, may be isolated by performing DLD using microfluidic devices that contain a channel through which fluid flows from an inlet at one end of the device to outlets at the opposite end. Basic principles of size based microfluidic separations and the design of obstacle arrays for separating cells have been provided elsewhere (see, US 2014/0342375; US 2016/0139012; U.S. Pat. Nos. 7,318,902 and 7,150,812), which are hereby incorporated herein in their entirety) and are also summarized in the sections below.

During DLD, a fluid sample containing cells is introduced into a device at an inlet and is carried along with fluid flowing through the device to outlets. As cells in the sample traverse the device, they encounter posts or other obstacles that have been positioned in rows and that form gaps or pores through which the cells must pass. Each successive row of obstacles is displaced relative to the preceding row so as to form an array direction that differs from the direction of fluid flow in the flow channel. The “tilt angle” defined by these two directions, together with the width of gaps between obstacles, the shape of obstacles, and the orientation of obstacles forming gaps are primary factors in determining a “critical size” for an array. Cells having a size greater than the critical size travel in the array direction, rather than in the direction of bulk fluid flow and particles having a size less than the critical size travel in the direction of bulk fluid flow. In devices used for leukapheresis-derived compositions, array characteristics may be chosen that result in white blood cells being diverted in the array direction whereas red blood cells and platelets continue in the direction of bulk fluid flow. In order to separate a chosen type of leukocyte from others having a similar size, a carrier may then be used that binds to that cell with in a way that promotes DLD separation and which thereby results in a complex that is larger than uncomplexed leukocytes. It may then be possible to carry out a separation on a device having a critical size smaller than the complexes but bigger than the uncomplexed cells.

The obstacles used in devices may take the shape of columns or be triangular, square, rectangular, diamond shaped, trapezoidal, hexagonal or teardrop shaped. In addition, adjacent obstacles may have a geometry such that the portions of the obstacles defining the gap are either symmetrical or asymmetrical about the axis of the gap that extends in the direction of bulk fluid flow.

### V. Making and Operating Microfluidic Devices

General procedures for making and using microfluidic devices that are capable of separating cells on the basis of size are well known in the art. Such devices include those described in U.S. Pat. Nos. 5,837,115; 7,150,812; 6,685,841; 7,318,902; 7,472,794; and U.S. Pat. No. 7,735,652; all of which are hereby incorporated by reference in their entirety. Other references that provide guidance that may be helpful in the making and use of devices for the present invention include: U.S. Pat. Nos. 5,427,663; 7,276,170; 6,913,697; 7,988,840; 8,021,614; 8,282,799; 8,304,230; 8,579,117; US 2006/0134599; US 2007/0160503; US 20050282293; US 2006/0121624; US 2005/0266433; US 2007/0026381; US 2007/0026414; US 2007/0026417; US 2007/0026415; US 2007/0026413; US 2007/0099207; US 2007/0196820; US 2007/0059680; US 2007/0059718; US 2007/005916; US 2007/0059774; US 2007/0059781; US 2007/0059719; US 2006/0223178; US 2008/0124721; US 2008/0090239; US 2008/0113358; and WO2012094642 all of which are also incorporated by reference herein in their entirety. Of the various references describing the making and use of devices, U.S. Pat. No. 7,150,812 provides particularly good guidance and U.S. Pat. No. 7,735,652 is of particular interest with respect to microfluidic devices for separations performed on samples with cells found in blood (in this regard, see also US 2007/0160503).

### VI. Making of CAR T Cells

The most preferred procedures described herein will lead to the making of CAR T cells. Methods for making and using CAR T cells have been described in, for example, U.S. Pat. Nos. 9,629,877; 9,328,156; 8,906,682; US 2017/0224789; US 2017/0166866; US 2017/0137515; US 2016/0361360; US 2016/0081314; US 2015/0299317; and US 2015/0024482; each of which is incorporated by reference herein in its entirety

### References

1. Blood 108:1903-1910 (2006)
2. Hematologica 98:1810-1818 (2013)
3. J. Pak. Med. Assoc. 66:1440-1443 (2016)
4. Blood 96:2469-2478 (2000)
5. Acta Biochimica Polonica 6:277-284 (2013)
6. J. Clin. Invest. 126:1612-1620 (2016)
7. J. Thromb. Haemost. 16: 316-329 (2018)
8. Nat. Chem. Bio.11(3):189-191 (2015)
9. Platelets 15:215-222 (2004)
10. J. of Vascular Surgery 48:715-722 (2008)
11. Br. J. Clin. Pharmacol. 48:197-199 (1999)
12. Thromb. and Hemost. 109:421-430 (2013)
13. Metallomics 8:91-100 (2016)
14. Molecular Biology of the Cell 12:2137-2145 (2001)
15. Nature Commun. 93767 (2018)
16. J. Immunol. 198:428-442 (2017)
17. Frontiers in Immunology 8:article 1849 (2018)
18. Frontiers in Immunology 10:article 963 (2019)
19. Circ. Res. 2019 Dec. 20. doi: 10.1161/CIRCRESAHA.119.315099. [Epub ahead of print]

All references cited herein are fully incorporated by reference. Having now fully described the invention, it will be understood by one of skill in the art that the invention may be performed within a wide and equivalent range of conditions, parameters and the like, without affecting the spirit or scope of the invention or any embodiment thereof.

What is claimed is:

1. A method of producing a population of therapeutically useful target cells from blood or a blood-related sample composition, comprising:
a) obtaining the blood or blood-related sample composition comprising said target cells;
b) during or after the obtaining of the blood or blood-related sample of step a), adjusting the sample so that it comprises one or more exogenous agents that inhibit platelet activation and/or inhibit the action of one or more factors that contribute to the proliferation or activation of the target cells;
c) separating the target cells from platelets to obtain an enriched population of target cells.

2. The method of claim 1, further comprising:
d) genetically engineering the target cells in the enriched population of cells obtained in step c), to produce genetically engineered target cells with a therapeutically useful phenotype.

3. The method of claim 2, wherein from the time that the blood or blood-related sample composition is obtained until the genetic engineering of cells is complete, the target cells have undergone no more than 2 doublings.

4. The method of any one of claims 1-3, wherein the ratio of non-terminally differentiated target cells to terminally differentiated target cells in the enriched population of target cells of step c) is at least 50% greater than in a sample composition obtained in the same way but in the absence of said exogenous agents.

5. The method of any one of claims 1-4, wherein the target cells after enrichment and/or after genetic transformation, are expanded in culture to obtain sufficient cells for the treatment of a patient.

6. The method of claim 5, wherein the target cells are T cells that have been genetically engineered to express chimeric antigen receptors.

7. The method of either claim 5 or 6, wherein after expansion in culture is complete, the target cells have undergone no more than 5 doublings.

8. The method of either claim 5 or 6, wherein after expansion in culture is complete, the target cells have undergone no more than 4 doublings.

9. The method of either claim 5 or 6, wherein after expansion in culture is complete, the target cells have undergone no more than 3 doublings.

10. The method of any one of claims 1-9, wherein the method is used in the production of a therapeutic product comprising target cells that have undergone no more than 5 doublings.

11. The method of any one of claims 1-9, wherein the method is used in the production of a therapeutic product comprising target cells that have undergone no more than 3 doublings.

12. The method of any one of claims 1-11, wherein the method is used in the production of a therapeutic product comprising target cells that, after administration to a patient, are capable of at least 10 doublings before reaching senescence.

13. The method of any one of claims 1-11, wherein the method is used in the production of a therapeutic product comprising target cells that, after administration to a patient, are capable of at least 15 doublings before reaching senescence.

14. The method of any one of claims 1-11, wherein the method is used in the production of a therapeutic product comprising target cells that, after administration to a patient, are capable of at least 17 doublings before reaching senescence.

15. The method of any one of claims 11-14, wherein the target cells in the therapeutic product are CAR T cells.

16. The method of any one of claims 1-15, wherein the blood or blood-related sample composition is a sample prepared by apheresis.

17. The method of any one of claims 1-15, wherein the blood or blood-related sample composition is a sample prepared by leukapheresis

18. The method of any one of claims 1-17, wherein the blood or blood-related sample composition has a white blood cell content of between 0.5×10⁹ to 13×10⁹ cells.

19. The method of any one of claims 1-18, wherein the exogenous agents are maintained in compositions containing the target cells up until at least the time the composition enhanced in target cells is obtained or at least until the time that the cells are genetically engineered has a white blood cell content of between 0.5×10⁹ to 13×10⁹ cells.

20. The method of any one of claims 1-19, wherein the exogenous agents are added at the time the sample composition is collected.

21. The method of any one of claims 1-20, wherein the one or more exogenous agents comprise an agent selected from the group consisting of: a gpIIb/IIIa inhibitor; tirofiban; GSK484; the non-permeable zinc-chelator DTPA; alexidine; Y-27632; MRS2578; rivaroxaban; Dextran-40; abciximab; Eptifibatide; Roxifiban; orbofiban; and combinations thereof.

22. The method of any one of claims 1-20, wherein the one or more exogenous agents comprise a gpIIb/IIIa inhibitor.

23. The method of any one of claims 1-20 wherein the sample composition of step b), and/or the enriched population of target cells of step c), comprises a gpIIb/IIIa inhibitor at concentration of from 0.5 μg/ml to 100 μg/ml.

24. The method of either claim 22 or claim 23, wherein the gpIIb/IIIa inhibitor is tirofiban.

25. The method of any one of claims 1-20, wherein the one or more exogenous agents comprise GSK484.

26. The method of any one of claims 1-20 wherein the sample composition of step b), and/or the enriched population of target cells of step c), comprises GSK484 at concentration of from 0.5 μg/ml to 100 μg/ml.

27. The method of any one of claims 1-20, wherein the one or more exogenous agents comprise the non-permeable zinc-chelator DTPA.

28. The method of any one of claims 1-20, wherein the sample composition of step b), and/or the enriched population of target cells of step c), comprises the non-permeable zinc-chelator DTPA at concentration of from 0.5 μg/ml to 100 μg/ml.

29. The method of any one of claims 1-20, wherein the one or more exogenous agents comprise alexidine.

30. The method of any one of claims 1-20, wherein the sample composition of step b), and/or the enriched population of target cells of step c), comprises alexidine at concentration of from 0.5 μg/ml to 100 μg/ml.

31. The method of any one of claims 1-20, wherein the one or more exogenous agents comprise Y-27632.

32. The method of any one of claims 1-20, wherein the sample composition of step b), and/or the enriched population of target cells of step c), comprises Y-27632 at concentration of from 0.5 μg/ml to 100 μg/ml.

33. The method of any one of claims 1-20, wherein the one or more exogenous agents comprise MRS2578.

34. The method of any one of claims 1-20, wherein the sample composition of step b), and/or the enriched population of target cells of step c), comprises MRS2578 at concentration of from 0.5 μg/ml to 100 μg/ml.

35. The method of any one of claims 1-20, wherein the sample composition of step b), and/or the enriched population of target cells of step b), comprises sodium citrate at concentration of 10 mM to 40 mM.

36. The method of any one of claims 1-20, wherein the one or more exogenous agents comprise rivaroxaban.

37. The method of any one of claims 1-20, wherein the sample composition of step a), and/or the enriched population of target cells of step b), comprises MRS2578 at concentration of from 0.5 μg/ml to 100 μg/ml.

38. The method of any one of claims 1-20, wherein the one or more exogenous agents comprise Dextran-40.

39. The method of any one of claims 1-20, wherein the sample composition of step a), and/or the enriched population of target cells of step b), comprises Dextran-40 at a concentration 0.1% to 10% by weight.
